(19) United States
(12) Patent Application Publication
(10) Pub. No.: US 2018/0228657 A1
(43) Pub. Date: Aug. 16, 2018

(54) SKIN PROTECTION SWEAT GUARD SYSTEMS

(71) Applicant: Jeannette Bedros, Los Angeles, CA (US)

(72) Inventor: Jeannette Bedros, Los Angeles, CA (US)

(21) Appl. No.: 15/950,164

(22) Filed: Apr. 10, 2018

Related U.S. Application Data
(63) Continuation-in-part of application No. 15/184,686, filed on Jun. 16, 2016.
(60) Provisional application No. 62/190,507, filed on Jul. 9, 2015.

(51) Int. Cl. A61F 13/02; A61F 13/06

(52) U.S. Cl. CPC A61F 13/0246; A61F 2013/00272; A61F 13/068; A61F 13/0223

(57) ABSTRACT

A skin protection sweat guard system having a thin, tissue-like exterior with an absorbent cotton interior which stops skin from becoming irritated when it rubs on other skin folds. It may be offered in a variety of shapes and sizes for all parts of the body. As designed it stays in place via the wearer's natural body moisture, without the need for adhesives. Provided are versions for between the toes, under the stomach, and under the breasts.

### CROSS-REFERENCE TO RELATED APPLICATION

The present application is a continuation in part of and is related to and claims priority from prior Provisional Application No. 62/190,507, filed Jul. 9, 2015, and pending U.S. Non-Provisional patent application Ser. No. 15/184,686, filed Jun. 16, 2016 which application is incorporated herein by reference.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of skin protection devices and more specifically relates to a skin protection sweat guard system.

### 2. Description of Related Art

When a person sweats and their skin rubs together with other skin on the body it can lead to redness, irritation, and discomfort. This is especially a problem for obese people. Diabetics are especially at risk of having open wounds exacerbated by folds of skin rubbing together. In public, it is not an option to simply wipe away the sweat, especially from intimate body locations. This is not desirable.

Several attempts have been made to solve the above-mentioned problems such as those found in U.S. Pub. No. 2011/0259349 to Tina McDonnell, U.S. Pat. No. 5,019,064 to Kasriel Eilender; U.S. Pat. No. 3,896,807 to Gilbert Buchalter; U.S. Pat. No. 3,260,261 to Matilda L Gallovich; U.S. Pat. No. 5,392,467 to Herbert Moretz; and U.S. Pat. No. 7,752,681 to Joyce Michel. This art is representative of skin protection devices. However, none of the above inventions and patents, taken either singly or in combination, is seen to describe the invention as claimed.

Preferably, a skin protection sweat guard system should provide a skin protection or sweat guard that may prevent skin irritation caused by sweat, folds of skin rubbing together, any area with excess moisture, and open wounds and, yet would operate reliably and be manufactured at a modest expense. Thus, a need exists for a reliable skin protection sweat guard system to avoid the above-mentioned problems.

### BRIEF SUMMARY OF THE INVENTION

In view of the foregoing disadvantages inherent in the known skin protection device art, the present invention provides a novel skin protection device. The general purpose of the present invention, which will be described subsequently in greater detail is to provide a skin protection sweat guard system.

A skin protection sweat guard system comprising a skin protection sweat guard member having a wicking-adhering pad having a body that is flexible comprising a front-surface, and a back-surface. The skin protection sweat guard system comprises the skin protection sweat guard member. The skin protection sweat guard member comprises the wicking-adhering pad, the wicking-adhering pad providing a wicking effect and an adhering effect when placed adjacent to skin when the skin is emitting perspiration.

The skin protection sweat guard member comprises adhesive. A removeable tab is included. When the removeable tab is removed, adhesive is exposed for adhering to a body. The wicking-adhering pad comprises the body. The wicking-adhering pad contours to a body-part of the user-wearer. Further, the wicking-adhering pad is deformable such that it is able to be wrapped about the body-part of the user-wearer. The wicking-adhering pad may comprise a planar-profile-rectangle-shape. The planar-profile-rectangular-shape comprises a cylindrical profile in a wrapped condition. The wicking-adhering pad is disposable.

The body comprises at least one layer for absorbency. The body is defined by the front-surface and the back-surface enclosing the at least one layer. The at least one layer is sandwiched within the wicking-adhering pad via the front-surface and the back-surface. The front-surface comprises cotton for absorbency and thereby providing comfort for the user-wearer. The cotton is a hypoallergenic and is non-irritating to the skin of the user-wearer. The back-surface is adjacent to the body-part of the user-wearer during an in-use condition. The skin protection sweat guard member is structured and arranged to provide a sweat guard that absorbs the perspiration and moisture between folds of the skin when adjacently placed via the adhering effect.

A kit is disclosed herein including the skin protection sweat guard system which may comprise a plurality of wicking-adhering pads comprising various shapes and sizes and a set of user instructions.

A method of using a skin protection sweat guard system, as described herein, may comprise the steps of: providing a skin protection sweat guard member having a wicking-adhering pad having a body that is flexible comprising, at least one wicking-absorbing layer; a front-surface layer, and a back-surface layer; removing the wicking-adhering pad from packaging; removing a removeable tab; applying wicking-adhering pad to a body-part of a user-wearer; wearing the wicking-adhering pad; and removing the wicking-adhering pad.

The present invention holds significant improvements and serves as a skin protection sweat guard system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, skin protection sweat guard systems, constructed and operative according to the teachings of the present invention.

FIG. 1 shows a perspective view illustrating a skin protection sweat guard system during an ‘in-use’ condition showing a wicking-adhering pad that is flexible according to an embodiment of the present invention.

FIG. 2 is a perspective view illustrating the skin protection sweat guard system comprising a skin protection sweat guard member on a foot of a user according to an embodiment of the present invention of FIG. 1.

FIG. 3 is a perspective view illustrating skin protection sweat guard member according to an embodiment of the present invention of FIGS. 1-2.

FIG. 4 is a perspective view illustrating skin protection sweat guard member comprising various shapes and sizes according to an embodiment of the present invention of FIGS. 1-3.

FIG. 5 is a flowchart illustrating a method of use for skin protection sweat guard system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a skin protection device and more particularly to a skin protection sweat guard system as used to improve skin protection and moisture wicking.

Generally speaking, skin protection sweat guard systems comprises a skin protection and sweat guard that absorbs sweat and moisture between folds of skin. This innovative product may comprise a disposable pad with a tissue-like exterior and absorbent cotton or similar material interior. The pad may be stored inside a sterile pouch and then disposed of after use. The pad may include a removeable adhesive configured to secure the pad to skin and release the wicking-adhering pad for removal.

Different shapes and styles may be provided to fit the needs of many different people, not just the obese or diabetic. The sweat guard also works well under uniforms, to absorb sweat and protect the skin from irritation. A U-shaped version may be provided including a U-shaped sweat guard specially designed for women under the breast, sweat causes irritation and blisters under the breast as well as who have had a breast mastectomy or constructive surgery. Most are sent home without proper dressing replacements. The sweat guard can be used to dress wounds as well. The U shape, or half moon shape, is the ideal pad to absorb sweat in an otherwise hard to reach area.

A square shaped embodiment may be included which is provided for athletes or other users to protect thighs from rubbing together while running, walking or exercising. The device eliminates discomfort, rashes and other unwanted issues associated with thighs rubbing together. A rectangular shaped version may further be provided to be used between layers of skin. As an example, the rectangular shaped embodiment may be used between skin fold of a stomach of a user. A toe ring embodiment may also be included in one embodiment. The toe ring sweat guard is configured to be worn around the toes to protect them from developing a fungus which could lead to amputation.

The sweat guard is made of disposable material and intended for single use only. The product is made of highly absorbent nonstick pads that cushion and protect without sticking to the area. The long lasting sure stick adhesive stays secure, while the soft, flexible fabric easily conforms to the body. The sweat guard may be provided in an easy to open wrapper. A user may simply remove the device from packaging, remove adhesive protector, and apply the pad to a desired area.

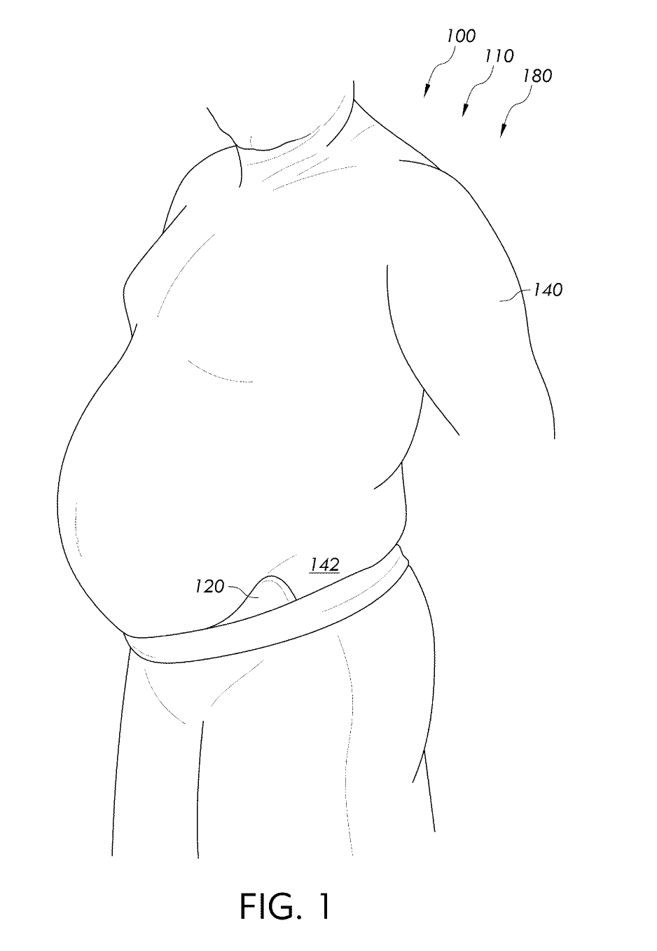

Referring to the drawings by numerals of reference there is shown in FIG. 1, skin protection sweat guard system 100 comprising skin protection sweat guard member 110 having wicking-adhering pad 120 having body 130 that is flexible comprising front-surface layer 134, and back-surface layer 136. Skin protection sweat guard system 100 comprises skin protection sweat guard member 110. Skin protection sweat guard member 110 comprises wicking-adhering pad 120. The wicking-adhering pad 120 provides a wicking effect and an adhering effect when placed adjacent to skin. The wicking-adhering pad 120 comprises adhesive on the back-surface 136 and is configured to adhere to skin 142 with the front-surface 134 opposing the skin 142 of a user-wearer 140 when applied.

Wicking-adhering pad 120 comprises body 130. Body 130 comprises at least one wicking-absorbing layer 152 for absorbency. Body 130 is defined by front-surface layer 134, and back-surface layer 136 enclosing the at least one wicking-absorbing layer 152. Skin protection sweat guard member 110 is structured and arranged to provide a sweat guard that absorbs perspiration and moisture between folds or layers of skin 142 when adjacently placed between folds or layers of skin 142 and adhered to skin 142 by the adhering effect of wicking-adhering pad 120.

Adhering effect of wicking-adhering pad 120 may be achieved by hydro-adhesion, which is well-known in the art of skin-adhering surface geometries. Wicking-adhering pad 120 includes adhesive and is structured and arranged such that adhesion is enabled when applied to skin 142 of user-wearer 140. The wicking-adhering pad 120 further includes a removeable tab 156 and adhesive. Front-surface 134 comprises a fluffed-contouring cotton for absorbency and thereby providing comfort for user-wearer 140. The fluffed-contouring cotton is a hypoallergenic and is non-irritating to skin 142 of user-wearer 140. The fluffed-contouring cotton is structured and arranged with wicking-adhering pad 120 such that wicking-adhering pad 120 contours to a body-part of user-wearer 140. Wicking-contouring pad 120 comprises a planar-profile-square-shape, wherein the planar-profile shape is configured such that wicking-adhering pad 120 can fit between two-toes of user-wearer 140 without inducing pain in user-wearer 140 or precluding user-wearer 140 from wearing user-wearer's 140 closed-toe shoes. Wicking-adhering pad 120 comprises a planar-profile-rectangle-shape. Wicking-adhering pad 120 comprises a planar-profile-arcuate-shape. Wicking-adhering pad 120 comprises a planar-profile-u-shape. Planar-profile-rectangular-shape comprises a cylindrical profile in a wrapped condition.

Skin protection sweat guard member 110 comprises a removeable adhesive configured to secure the wicking-adhering pad 120 to skin and release the wicking-adhering pad 120 for removal. At least one wicking-absorbing layer 152 is sandwiched within wicking-adhering pad 120 via front-surface layer 134 and back-surface layer 136. Back-surface layer 136 is adjacent to body-part of user-wearer 140 during an in-use condition. Wicking-adhering pad 120 is deformable such that it is able to be wrapped about body-part of user-wearer 140. The wicking-adhering pad 120 is disposable.

It should be noted that skin protection sweat guard system 100 may be sold as kit 440 comprising the following parts: at least one wicking-adhering pad 120; and at least one set of user instructions. The kit has instructions such that functional relationships are detailed in relation to the structure of the invention (such that the invention can be used, maintained, or the like in a preferred manner). Skin protection sweat guard system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

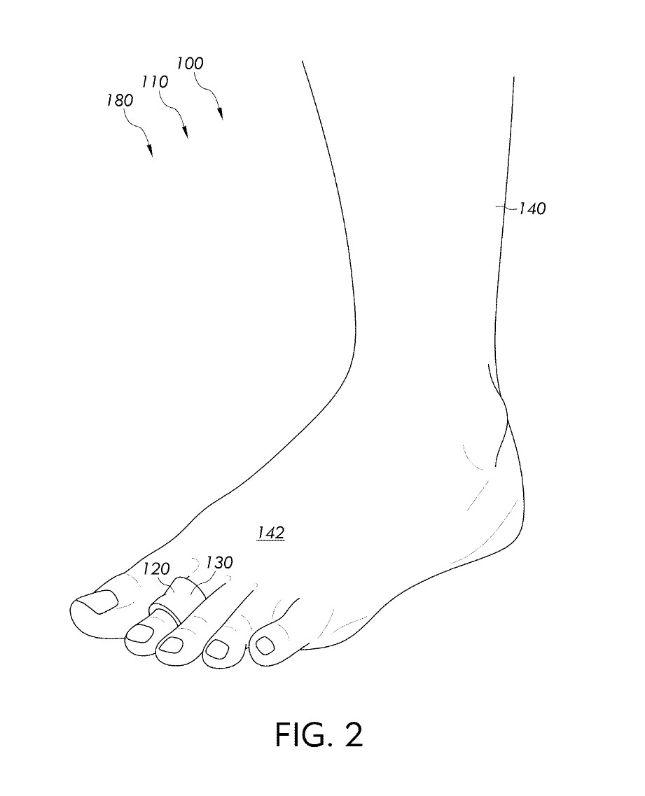

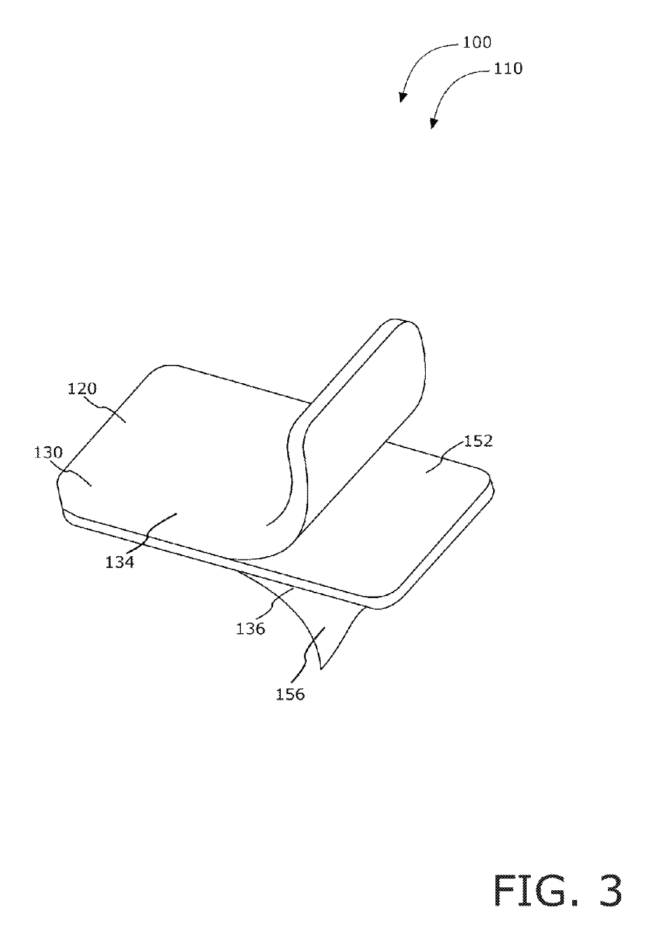

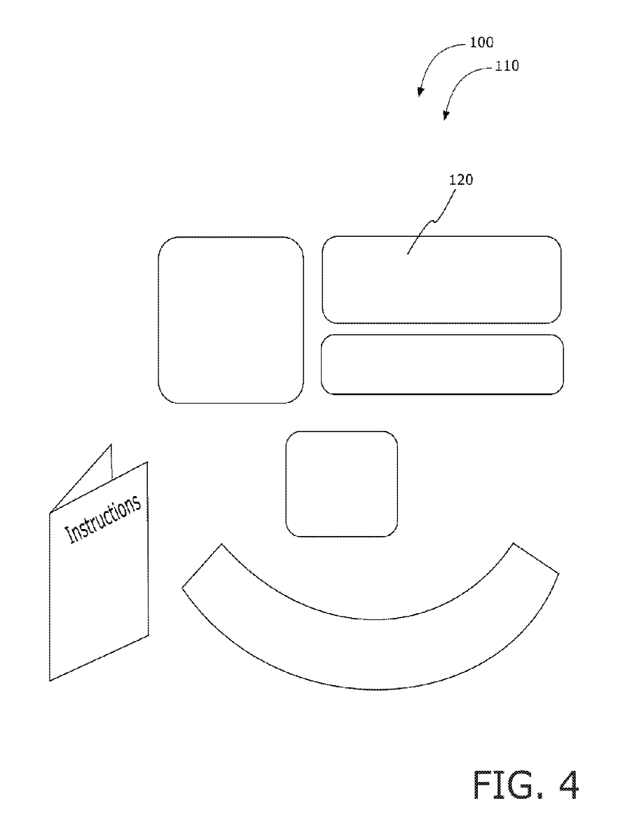

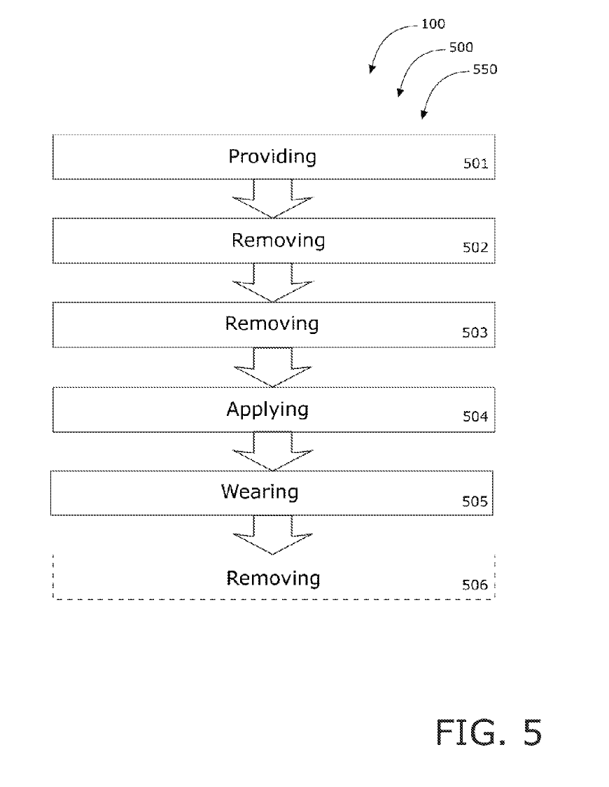

Referring now to FIG. 5 showing flowchart 550 illustrating method of use 500 for skin protection sweat guard systems 100 according to an embodiment of the present invention of FIGS. 1-4. As shown, method of use 500 may comprise the steps of: step one 501, providing a skin protection sweat guard member 110 having a wicking-adhering pad 120 having a body 130 that is flexible comprising at least one wicking-absorbing layer 152; a front-surface layer 134, and a back-surface layer 136; step two 502, removing the wicking-adhering pad 120 from packaging; step three 503, removing a removeable tab 156; step four 504, applying wicking-adhering pad 120 to a body-part of a user-wearer 140; and step five 505, wearing the wicking-adhering pad 120; step six 506, removing the wicking-adhering pad.

It should be noted that step six 506 is an optional step and may not be implemented in all cases. Optional steps of method of use 500 are illustrated using dotted lines in FIG. 5 so as to distinguish them from the other steps of method of use 500.

It should be noted that the steps described in the method of use can be carried out in many different orders according to user preference. The use of “step of” should not be interpreted as “step for”, in the claims herein and is not intended to invoke the provisions of 35 U.S.C. § 112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A skin protection sweat guard system comprising:
a skin protection sweat guard member having,
a wicking-adhering pad having,
a body that is flexible comprising,
at least one wicking-absorbing layer;
a front-surface layer, and
a back-surface layer;
wherein said skin protection sweat guard member comprises said wicking-adhering pad, said wicking-adhering pad providing a wicking effect when placed adjacent to skin;
wherein said wicking-adhering pad comprises adhesive on a back-surface configured to adhere to said skin, said front-surface is opposing said skin of a user-wearer when applied;
wherein said at least one wicking-absorbing layer is structured and arranged with said front-surface layer and further structured and arranged with said back-surface layer to provide said wicking effect and said adhering effect when said skin protection sweat guard member is placed adjacent to said skin of a user-wearer, whereby establishing a sweat guard that absorbs the perspiration between folds of skin.

2. The skin protection sweat guard system of claim 1 wherein said wicking-adhering pad further comprises a removeable tab.

3. The skin protection sweat guard system of claim 2 wherein said removeable tab exposes said adhesive when removed.

4. The skin protection sweat guard system of claim 3 wherein said front-surface comprises fluffed-contouring cotton that is structured and arranged with said wicking-adhering pad such that said wicking-adhering pad contours to a body-part of the user-wearer for absorbing the perspiration and thereby providing comfort for the user-wearer.

5. The skin protection sweat guard system of claim 4 wherein said cotton is a hypoallergenic and is non-irritating to said skin of said user-wearer.

6. The skin protection sweat guard system of claim 1 wherein said wicking-adhering pad comprises a planar-profile-square-shape, wherein said planar-profile shape is configured such that said wicking-adhering pad can fit between two-toes of the user-wearer without inducing pain in the user-wearer or precluding the user-wearer from the wearing user-wearer's closed-toe shoes.

7. The skin protection sweat guard system of claim 6 wherein wicking-adhering pad comprises a planar-profile-square-shape.

8. The skin protection sweat guard system of claim 6 wherein said wicking-adhering pad comprises a planar-profile-rectangle-shape.

9. The skin protection sweat guard system of claim 6 wherein said wicking-adhering pad comprises a planar-profile-arcuate-shape.

10. The skin protection sweat guard system of claim 6 wherein said wicking-adhering pad comprises a planar-profile-u-shape.

11. The skin protection sweat guard system of claim 8 wherein said planar-profile-rectangular-shape comprises a cylindrical profile in a wrapped condition.

12. The skin protection sweat guard system of claim 3 wherein said skin protection sweat guard member comprises a removeable said adhesive configured to secure said wicking-adhering pad to skin and release said wicking-adhering pad for removal.

13. The skin protection sweat guard system of claim 1 wherein said at least one layer is sandwiched within said wicking-adhering pad via said front-surface layer and said back-surface layer.

14. The skin protection sweat guard system of claim 6 wherein said back-surface is adjacent to the body-part of the user-wearer during an in-use condition.

15. The skin protection sweat guard system of claim 6 wherein said wicking-adhering pad is deformable such that it is able to be wrapped about the body-part of the user-wearer.

16. The skin protection sweat guard system of claim 1 wherein said pad is disposable.

17. A skin protection sweat guard system comprising:
a skin protection sweat guard member having,
a wicking-adhering pad having,
a body that is flexible comprising,
at least one wicking-absorbing layer;
a front-surface layer, and
a back-surface layer;
wherein said skin protection sweat guard member comprises said wicking-adhering pad, said wicking-adhering pad providing a wicking effect when placed adjacent to skin;
wherein said wicking-adhering pad comprises adhesive on a back-surface configured to adhere to said skin, said front-surface is opposing said skin of a user-wearer when applied;
wherein said pad further comprises a removeable tab;
wherein said removeable tab exposes said adhesive when removed;
wherein said skin protection sweat guard member comprises a removeable said adhesive configured to secure said pad to skin and release said pad for removal;
wherein said wicking-adhering pad contours to a body-part of said user-wearer;
wherein said wicking-adhering pad is deformable such that it is able to be wrapped about said body-part of said user-wearer;
wherein said wicking-adhering pad comprises a planar-profile-rectangle-shape;
wherein said planar-profile-rectangular-shape comprises a cylindrical profile in a wrapped condition;
wherein said wicking-adhering pad is disposable;
wherein said at least one layer is sandwiched within said wicking-adhering pad via said front-surface and said back-surface;
wherein said front-surface comprises cotton for absorbency and thereby providing comfort for said user-wearer;
wherein said cotton is a hypoallergenic and is non-irritating to said skin of said user-wearer;
wherein said back-surface is adjacent to said body-part of said user-wearer during an in-use condition; and
wherein said at least one wicking-absorbing layer is structured and arranged with said front-surface layer and further structured and arranged with said back-surface layer to provide said wicking effect and said adhering effect when said skin protection sweat guard member is placed adjacent to said skin of a user-wearer, whereby establishing a sweat guard that absorbs the perspiration between folds of skin.

18. The skin protection sweat guard system of claim 17 further comprising a kit including:
a plurality of wicking-adhering pads comprising various shapes and sizes; and
a set of user instructions.

19. A method of using a skin protection sweat guard system comprising the steps of:
providing a skin protection sweat guard member having,
a wicking-adhering pad having,
a body that is flexible comprising,
at least one wicking-absorbing layer;
a front-surface layer, and
a back-surface layer;
removing said wicking-adhering pad from packaging;
removing a removeable tab;
applying wicking-adhering pad to a body-part of a user-wearer; and
wearing said wicking-adhering pad.

20. The method of claim 19 further comprising the step of:
removing said wicking-adhering pad.
